(19) United States
(12) Patent Application Publication
(10) Pub. No.: US 2020/0376047 A1
(43) Pub. Date: Dec. 3, 2020

(54) METHODS FOR THE TREATMENT OF INFLAMMATION AND INFLAMMATORY CONDITIONS

(71) Applicant: Servatus Ltd, Sippy Downs, QLD (AU)

(72) Inventor: Wayne Finlayson, Peregian Springs, Queensland (AU)

(21) Appl. No.: 16/604,325

(22) PCT Filed: Apr. 11, 2018
(86) PCT No.: PCT/AU2018/050326

§ 371 Date: Oct. 10, 2019

(30) Foreign Application Priority Data
Apr. 11, 2017 (AU) 2017901320
Jun. 15, 2017 (AU) 2017902271

(51) Int. Cl. A61K 35/747; A61P 29/00; A61P 1/00; A61P 17/04; A61P 17/02

(52) U.S. Cl. CPC A61K 35/747; A61P 29/00; A61P 17/02; A61P 17/04; A61P 1/00

(57) ABSTRACT

Provided herein are methods for treating or preventing inflammation, or an inflammatory or autoimmune condition, methods for treating or preventing a skin or nail condition or infection, methods for treating or preventing symptoms of gastrointestinal infections, and methods for promoting wound healing. Embodiments of the methods comprise administering a combination of two or more strains of Lactobacillus selected from Lactobacillus buchneri, Lactobacillus zeae, Lacobacillus paracasei, Lactobacillus parafarraginis, and Lactobacillus rapi, and/or one or more culture supernatants or cell free filtrates derived from culture media in which one or more of said strains has been cultured.

### FIELD OF THE ART

The present disclosure relates generally to methods for the treatment of inflammation and inflammatory and autoimmune conditions. Also provided are methods for promoting wound healing. The methods disclosed herein comprise the administration of two or more microbial strains, culture supernatants or cell free filtrates to mammalian subjects in need thereof.

### BACKGROUND

Inflammation is a normal response mechanism assisting in protecting the body from infection and injury. However abnormal or uncontrolled inflammatory responses can result in the development of acute or chronic inflammatory and autoimmune disorders or conditions. Chronic inflammatory and autoimmune conditions can be debilitating and cause enormous discomfort and pain to sufferers. Moreover such conditions are increasing in prevalence as populations around the world age.

Steroids have been the primary therapeutic anti-inflammatory agent relied upon for many decades. More recently non-steroidal anti-inflammatory drugs (NSAIDs) have begun to be commonly employed to manage or treat inflammation. However, the continued use of such agents comes with significant disadvantages and side effects. For example, associated with continued steroid use are significant side effects including stomach ulcers and bleeding. Additionally, it is well known that NSAIDs produce lesions in the gastrointestinal tract, depending on the length of the treatment and on the type of drug. This problem is of particular importance in cases where the therapy must be protracted for a long time, such as in the treatment of chronic inflammatory disorders where long term treatment is needed to manage the inflammatory state and associated pain.

There is a continuing need for the development of new and improved therapeutic options for the treatment of inflammation, and inflammatory and autoimmune conditions.

### SUMMARY OF THE DISCLOSURE

One aspect of the present disclosure provides a method for treating or preventing inflammation, or an inflammatory or autoimmune condition, in a subject, the method comprising administering to the subject a combination of two or more strains of Lactobacillus selected from Lactobacillus buchneri, Lactobacillus zeae, Lacobacillus paracasei, Lactobacillus parafarraginis, and Lactobacillus rapi, and/or one or more culture supernatants or cell free filtrates derived from culture media in which one or more of said strains has been cultured.

In a particular embodiment the inflammation is gastrointestinal inflammation or inflammation of the joints. The gastrointestinal inflammation may be, for example, gastritis or gastroenteritis. The method may be employed to treat or prevent one or more symptoms of a gastrointestinal infection, such as food poisoning. The gastrointestinal infection may be a bacterial, viral or parasitic infection. The at least one symptom may be diarrhoea, poor stool consistency, or faecal blood presence, including faceal occult blood.

The inflammation of the joints may be, or be associated with arthritis. The arthritis may be, for example, rheumatoid arthritis or osteoarthritis.

In a particular embodiment the inflammatory condition is an inflammatory gastrointestinal condition or an inflammatory skin condition. The gastrointestinal condition may be an inflammatory bowel disease. The inflammatory bowel disease may be, for example, irritable bowel syndrome or colitis, such as ulcerative colitis or Crohn's disease.

The inflammation may be associated with a skin or nail condition or infection and/or an inflammatory skin condition. The skin condition may be, for example, psoriasis, dermatitis, eczema, rosacea, acne, ichtyosis, or other skin condition characterized by or associated with inflammation, plaques or skin lesions. The skin or nail condition or infection may be a fungal skin or nail infection such as tinea. Exemplary fungal infections include tinea pedis (Athlete's foot), tinea cruris (tinea of the groin, Jock itch), tinea capitis (tinea of the head and scalp), tinea corporis (tinea of the body) and tinea unguium (tinea of a fingernail or toenail, onychomycosis).

In exemplary embodiments, for the treatment of gastrointestinal inflammation, inflammation of the joints, or of associated inflammatory conditions, the combination may be administered orally or sublingually. In exemplary embodiments, for the treatment of skin inflammation, inflammation of the joints, or of associated conditions, the combination may be administered topically.

The method may comprise the administration of a combination of strains of two, three, four or all of said Lactobacillus species or one or more culture supernatants or cell free filtrates derived from culture media in which one or more of said strains has been cultured. The combination may represent a synergistic combination.

The Lactobacillus buchneri strain may be Lactobacillus buchneri Lb23. In a particular embodiment the Lactobacillus buchneri strain is Lactobacillus buchneri Lb23 deposited under Accession Number V11/022946.

The Lactobacillus zeae strain may be Lactobacillus zeae Lz26. In a particular embodiment the Lactobacillus zeae strain is Lactobacillus zeae Lz26 deposited under Accession Number V11/022948.

The Lactobacillus paracasei may be Lactobacillus paracasei Lp9. In a particular embodiment the Lactobacillus paracasei strain is Lactobacillus paracasei Lp9 deposited under Accession Number V12/022849.

The Lactobacillus parafarraginis strain may be Lactobacillus parafarraginis Lp18. In a particular embodiment the Lactobacillus parafarraginis strain is Lactobacillus parafarraginis Lp18 deposited under Accession Number V11/022945.

The Lactobacillus rapi strain may be Lactobacillus rapi Lr24. In a particular embodiment the Lactobacillus rapi strain is Lactobacillus rapi Lr24 deposited under Accession Number V11/022947.

In an exemplary embodiment, the method comprises the administration of a combination of Lactobacillus buchneri, Lactobacillus zeae and Lacobacillus paracasei, and/or one or more culture supernatants or cell free filtrates derived from culture media in which one or more of said strains has been cultured. In a further exemplary embodiment, the method comprises the administration of a combination of Lactobacillus buchneri Lb23, Lactobacillus zeae Lz26 and Lacobacillus paracasei Lp9, and/or one or more culture supernatants or cell free filtrates derived from culture media in which one or more of said strains has been cultured.

The method may further comprise administration of a strain of Acetobacter fabarum or a culture supernatant or cell free filtrate derived from culture media in which said strain has been cultured. The Acetobacter fabarum strain may be Acetobacter fabarum Af15. In a particular embodiment the Acetobacter fabarum strain is Acetobacter fabarum Af15 deposited under Accession Number V11/022943.

The method may further comprise administration of a yeast strain or a culture supernatant or cell free filtrate derived from culture media in which said strain has been cultured. The yeast may be a strain of Candida ethanolica. The Candida ethanolica strain may be Candida ethanolica Ce31. In a particular embodiment the Candida ethanolica strain is Candida ethanolica Ce31 deposited under Accession Number V11/022944.

In an exemplary embodiment a combination of Lactobacillus parafarraginis, Lactobacillus buchneri, Lactobacillus rapi, Lactobacillus zeae, Acetobacter fabarum and Candida ethanolica, and/or one or more culture supernatants or cell free filtrates derived from culture media in which one or more of said strains has been cultured, is administered. In a further exemplary embodiment a combination of Lactobacillus parafarraginis Lp18, Lactobacillus buchneri Lb23, Lactobacillus rapi Lr24, Lactobacillus zeae Lz26, Acetobacter fabarum Af15 and Candida ethanolica Ce31, and/or one or more culture supernatants or cell free filtrates derived from culture media in which one or more of said strains has been cultured, is administered.

One or more of the strains may be encapsulated. Where multiple strains are encapsulated, the strains may be individually encapsulated or combined in a single encapsulation.

In particular embodiments the at least one strain, culture supernatant(s) or cell free filtrate(s) is administered in the form of a pharmaceutically acceptable composition.

The method may further comprise administering to the subject an effective amount of one or more antimicrobial agents.

A further aspect of the present disclosure provides a method for treating or preventing a skin or nail condition or infection, the method comprising administering to a subject in need thereof a combination of two or more strains of Lactobacillus selected from Lactobacillus buchneri, Lactobacillus zeae, Lacobacillus paracasei, Lactobacillus parafarraginis, and Lactobacillus rapi, and/or one or more culture supernatants or cell free filtrates derived from culture media in which one or more of said strains has been cultured.

The skin or nail condition or infection may be, for example, psoriasis, dermatitis, eczema, rosacea, acne, ichtyosis, or other skin condition characterized by or associated with inflammation, plaques or skin lesions. The skin and/or nail condition may be a fungal skin or nail infection such as tinea. Exemplary fungal infections include tinea pedis (Athlete's foot), tinea cruris (tinea of the groin, Jock itch), tinea capitis (tinea of the head and scalp), tinea corporis (tinea of the body) and tinea unguium (tinea of a fingernail or toenail, onychomycosis).

A further aspect of the present disclosure provides a method for treating or preventing at least one symptom of a gastrointestinal infection, the method comprising administering to a subject in need thereof a combination of two or more strains of Lactobacillus selected from Lactobacillus buchneri, Lactobacillus zeae, Lacobacillus paracasei, Lactobacillus parafarraginis, and Lactobacillus rapi, and/or one or more culture supernatants or cell free filtrates derived from culture media in which one or more of said strains has been cultured.

The gastrointestinal infection may be a bacterial, viral or parasitic infection. The at least one symptom may be diarrhoea, poor stool consistency, or faecal blood presence, including faceal occult blood.

A further aspect of the present disclosure provides a method for treating or preventing inflammation, or an inflammatory or autoimmune condition, in a subject, the method comprising administering to the subject a combination of microbial strains comprising strains of Lactobacillus buchneri and Lactobacillus zeae, and/or one or more culture supernatants or cell free filtrates derived from culture media in which one or more of said strains has been cultured.

A further aspect of the present disclosure provides a method for treating or preventing a skin or nail condition or infection, the method comprising administering to a subject in need thereof a combination of microbial strains comprising strains of Lactobacillus buchneri and Lactobacillus zeae, and/or one or more culture supernatants or cell free filtrates derived from culture media in which one or more of said strains has been cultured.

A further aspect of the present disclosure provides a method for treating or preventing at least one symptom of a gastrointestinal infection, the method comprising administering to a subject in need thereof a combination of microbial strains comprising strains of Lactobacillus buchneri and Lactobacillus zeae, and/or one or more culture supernatants or cell free filtrates derived from culture media in which one or more of said strains has been cultured.

A further aspect of the present disclosure provides a method for treating or preventing inflammation, or an inflammatory or autoimmune condition, in a subject, the method comprising administering to the subject a combination of microbial strains comprising strains of Lactobacillus buchneri, Lactobacillus zeae and Lactobacillus paracasei, and/or one or more culture supernatants or cell free filtrates derived from culture media in which one or more of said strains has been cultured.

A further aspect of the present disclosure provides a method for treating or preventing a skin or nail condition or infection, the method comprising administering to a subject in need thereof a combination of microbial strains comprising strains of Lactobacillus buchneri, Lactobacillus zeae and Lactobacillus paracasei, and/or one or more culture supernatants or cell free filtrates derived from culture media in which one or more of said strains has been cultured.

A further aspect of the present disclosure provides a method for treating or preventing at least one symptom of a gastrointestinal infection, the method comprising administering to a subject in need thereof a combination of microbial strains comprising strains of Lactobacillus buchneri, Lactobacillus zeae and Lactobacillus paracasei, and/or one or more culture supernatants or cell free filtrates derived from culture media in which one or more of said strains has been cultured.

A further aspect of the present disclosure provides a method for treating or preventing inflammation, or an inflammatory or autoimmune condition, in a subject, the method comprising administering to the subject a combination of microbial strains comprising strains of Lactobacillus parafarraginis, Lactobacillus buchneri, Lactobacillus rapi, Lactobacillus zeae, Acetobacter fabarum and Candida ethanolica, and/or one or more culture supernatants or cell free filtrates derived from culture media in which one or more of said strains has been cultured.

A further aspect of the present disclosure provides a method for treating or preventing a skin or nail condition or infection, the method comprising administering to a subject in need thereof a combination of microbial strains comprising strains of Lactobacillus parafarraginis, Lactobacillus buchneri, Lactobacillus rapi, Lactobacillus zeae, Acetobacter fabarum and Candida ethanolica, and/or one or more culture supernatants or cell free filtrates derived from culture media in which one or more of said strains has been cultured.

A further aspect of the present disclosure provides a method for treating or preventing at least one symptom of a gastrointestinal infection, the method comprising administering to a subject in need thereof a combination of microbial strains comprising strains of Lactobacillus parafarraginis, Lactobacillus buchneri, Lactobacillus rapi, Lactobacillus zeae, Acetobacter fabarum and Candida ethanolica, and/or one or more culture supernatants or cell free filtrates derived from culture media in which one or more of said strains has been cultured.

A further aspect of the present disclosure provides a method for promoting wound healing in a subject, the method comprising administering to the subject a combination of two or more strains of Lactobacillus selected from Lactobacillus buchneri, Lactobacillus zeae, Lacobacillus paracasei, Lactobacillus parafarraginis, and Lactobacillus rapi, and/or one or more culture supernatants or cell free filtrates derived from culture media in which one or more of said strains has been cultured.

A further aspect of the present disclosure provides a method for promoting wound healing in a subject, the method comprising administering to the subject a combination of microbial strains comprising strains of Lactobacillus buchneri and Lactobacillus zeae, and/or one or more culture supernatants or cell free filtrates derived from culture media in which one or more of said strains has been cultured.

A further aspect of the present disclosure provides a method for promoting wound healing in a subject, the method comprising administering to the subject a combination of microbial strains comprising strains of Lactobacillus buchneri, Lactobacillus zeae and Lactobacillus paracasei, and/or one or more culture supernatants or cell free filtrates derived from culture media in which one or more of said strains has been cultured.

A further aspect of the present disclosure provides a method for promoting wound healing in a subject, the method comprising administering to the subject a combination of microbial strains comprising strains of Lactobacillus parafarraginis, Lactobacillus buchneri, Lactobacillus rapi, Lactobacillus zeae, Acetobacter fabarum and Candida ethanolica, and/or one or more culture supernatants or cell free filtrates derived from culture media in which one or more of said strains has been cultured.

In accordance with the above aspects and embodiments, the methods may comprise administering to the subject a probiotic composition comprising a combination of said microbial strains. The probiotic composition may be administered in the form of a food or beverage.

Also provided herein is the use of two or more strains of Lactobacillus, wherein the Lactobacillus is selected from Lactobacillus parafarraginis, Lactobacillus buchneri, Lactobacillus rapi and Lactobacillus zeae, and/or one or more culture supernatants or cell free filtrates derived from culture media in which one or more of said strains has been cultured, for the manufacture of a composition for treating or preventing inflammation, an inflammatory or autoimmune condition, a skin or nail condition or infection, or at least one symptom of a gastrointestinal infection.

Also provided herein is the use of two or more strains of Lactobacillus, wherein the Lactobacillus is selected from Lactobacillus parafarraginis, Lactobacillus buchneri, Lactobacillus rapi and Lactobacillus zeae, and/or one or more culture supernatants or cell free filtrates derived from culture media in which one or more of said strains has been cultured, for the manufacture of a composition for promoting wound healing.

### BRIEF DESCRIPTION OF THE DRAWINGS

Exemplary embodiments of the present disclosure are described herein, by way of non-limiting example only, with reference to the following drawings.

FIG. 1. Faecal blood occurrence scores in mice of a DSS-induced model of acute colitis (as described in Example 3) between days 1 and 11 following treatment. From left to right, Group 1, vehicle only negative control; Group 2, 3% DSS+vehicle; Group 3, 3% DSS+formulation as described in Example 3 at 10⁶ cfu/ml of each species; Group 4, 3% DSS+formulation as described in Example 3 at 10⁹ cfu/ml of each species. **, p<0.01 Dunnett's test compared to Group 2.

FIG. 2. Combined faecal blood occurrence and stool consistency scores in mice of a DSS-induced model of acute colitis (as described in Example 3) between days 1 and 11 following treatment. From left to right, Group 1, vehicle only negative control; Group 2, 3% DSS+vehicle; Group 3, 3% DSS+formulation as described in Example 3 at 10⁶ cfu/ml of each species; Group 4, 3% DSS+formulation as described in Example 3 at 10⁹ cfu/ml of each species. ***, p<0.001 Dunnett's test compared to Group 2.

### DETAILED DESCRIPTION

Unless defined otherwise, all technical and scientific terms used herein have the same meaning as commonly understood by those of ordinary skill in the art to which the disclosure belongs. Although any methods and materials similar or equivalent to those described herein can be used in the practice or testing of the present disclosure, typical methods and materials are described.

The articles “a” and “an” are used herein to refer to one or to more than one (i.e., to at least one) of the grammatical object of the article. By way of example, “an element” means one element or more than one element.

In the context of this specification, the term “about,” is understood to refer to a range of numbers that a person of skill in the art would consider equivalent to the recited value in the context of achieving the same function or result.

Throughout this specification and the claims which follow, unless the context requires otherwise, the word “comprise”, and variations such as “comprises” or “comprising”, will be understood to imply the inclusion of a stated integer or step or group of integers or steps but not the exclusion of any other integer or step or group of integers or steps.

As used herein the term “effective amount” includes within its meaning a non-toxic but sufficient amount of composition to provide the desired therapeutic effect. The exact amount required will vary from subject to subject depending on factors such as the species being treated, the age and general condition of the subject, the severity of the inflammatory or autoimmune condition being treated, the particular agent being administered and the mode of administration and so forth. For any given case, an appropriate “effective amount” may be determined by one of ordinary skill in the art using only routine experimentation.

The term “subject” as used herein refers to mammals and includes humans, primates, livestock animals (e.g. cattle, dairy cows, horses, sheep, pigs), laboratory test animals (e.g. mice, rabbits, rats, guinea pigs), companion animals (e.g. dogs, cats), performance animals (e.g. racehorses), and captive wild animals. In exemplary embodiments, the mammal is human or a livestock animal.

As used herein the terms “treating”, “treatment” and the like refer to any and all applications which remedy, or otherwise hinder, retard, or reverse the progression of, inflammation or of a condition, or at least one symptom of such a condition, including reducing the severity of a condition. Thus, treatment does not necessarily imply that a subject is treated until complete elimination of, or recovery from, the condition. Similarly, the terms “preventing”, “prevention” and the like refer to any and all applications which prevent the establishment of an inflammatory or autoimmune condition or otherwise delay the onset of such a condition.

The term “optionally” is used herein to mean that the subsequently described feature may or may not be present or that the subsequently described event or circumstance may or may not occur. Hence the specification will be understood to include and encompass embodiments in which the feature is present and embodiments in which the feature is not present, and embodiments in which the event or circumstance occurs as well as embodiments in which it does not.

In the context of this specification, the term “probiotic” is to be given its broadest construction and is understood to refer to a microbial cell population or preparation, or component of a microbial cell population or preparation, which when administered to a subject in an effective amount promotes a health benefit in the subject.

In the context of this specification, the term “prebiotic” is to be given its broadest construction and is understood to refer to any non-digestible substance that stimulates the growth and/or activity of probiotic bacteria in the digestive system.

In the context of this specification, the terms “food”, “foods”, “beverage” or “beverages” include but are not limited to health foods and beverages, functional foods and beverages, and foods and beverages for specified health use. When such foods or beverages of the present invention are used for subjects other than humans, the terms can be used to include a feedstuff.

Provided herein are methods for treating or preventing inflammation, an inflammatory or autoimmune condition or a skin or nail condition or infection, comprising administering to a subject in need thereof a combination of two or more strains of Lactobacillus selected from Lactobacillus buchneri, Lactobacillus zeae, Lacobacillus paracasei, Lactobacillus parafarraginis, and Lactobacillus rapi, and/or one or more culture supernatants or cell free filtrates derived from culture media in which one or more of said strains has been cultured.

Also provided herein are methods for treating or preventing inflammation, an inflammatory or autoimmune condition or a skin or nail condition or infection, comprising administering to a subject in need thereof a combination of microbial strains comprising strains of Lactobacillus buchneri and Lactobacillus zeae, and/or one or more culture supernatants or cell free filtrates derived from culture media in which one or more of said strains has been cultured.

Also provided herein are methods for treating or preventing inflammation, an inflammatory or autoimmune condition or a skin or nail condition or infection, comprising administering to a subject in need thereof a combination of microbial strains comprising strains of Lactobacillus buchneri, Lactobacillus zeae and Lactobacillus paracasei, and/or one or more culture supernatants or cell free filtrates derived from culture media in which one or more of said strains has been cultured.

Also provided herein are methods for treating or preventing inflammation, an inflammatory or autoimmune condition or a skin or nail condition or infection, comprising administering to a subject in need thereof a combination of microbial strains comprising strains of Lactobacillus parafarraginis, Lactobacillus buchneri, Lactobacillus rapi, Lactobacillus zeae, Acetobacter fabarum and Candida ethanolica, and/or one or more culture supernatants or cell free filtrates derived from culture media in which one or more of said strains has been cultured.

As used herein, the term “inflammatory condition” typically refers to a condition characterised by inflammation, or the complex biological response to a noxious stimulus such as infection by a microbial pathogen and/or virus. The clinical features of an inflammatory condition is likely to depend on the noxious stimulus (or stimuli), but may be characterised by heat, pain, redness or swelling of the affected organ or tissue. The inflammatory condition may be acute or chronic.

In particular embodiments the inflammation may be gastrointestinal inflammation or inflammation of the joints. The gastrointestinal inflammation may be, for example, gastritis or gastroenteritis. The inflammation of the joints may be, or be associated with arthritis. The arthritis may be, for example, rheumatoid arthritis or osteoarthritis.

In other particular embodiments, the condition may be a gastrointestinal condition or a skin or nail condition or infection. The gastrointestinal condition may be an inflammatory bowel disease. The inflammatory bowel disease may be, for example, colitis, or irritable bowel syndrome. The colitis may be, for example, ulcerative colitis, Crohn's disease, ischemic colitis or Clostridium difficile (C. difficile) colitis. The skin condition may be, for example, psoriasis, dermatitis, eczema, rosacea, acne, ichtyosis, fungal skin and/or nail infection or other skin condition characterized by or associated with inflammation, plaques or skin lesions. Exemplary forms of psoriasis include plaque psoriasis, guttate psoriasis and pustular psoriasis. Exemplary forms of dermatitis include atopic dermatitis, infant dermatitis, seborrhoic dermatitis, contact dermatitis, occupational dermatitis, hand dermatitis, nummular dermatitis, stasis dermatitis, perioral dermatitis and dermatitis herpetiformis. Exemplary fungal infections include tinea pedis (Athlete's foot), tinea cruris (tinea of the groin, Jock itch), tinea capitis (tinea of the head and scalp), tinea corporis (tinea of the body) and tinea unguium (tinea of a fingernail or toenail, onychomycosis).

Other exemplary inflammatory or autoimmune conditions include, for example, sinusitis, allergic disorders such as asthma, chronic fatigue syndrome, systemic lupus erythematosus, Sjögren's syndrome, inflammation of the prostate, inflammation of the urinary tract, pelvic inflammatory disease, pancreatitis, vasculitis, inflammation of the feet including gout, period pain.

As exemplified herein, a combination of Lactobacillus buchneri, Lactobacillus zeae and Lactobacillus paracasei significantly improved stool consistency and significantly reduced faecal blood occurrence in a mouse model of colitis. Accordingly, embodiments of the present disclosure provide methods for treating or preventing at least one symptom of a gastrointestinal infection, such as a bacterial infection (e.g Salmonella, E. coli, Listeria, B. cereus), viral infection (e.g. norovirus, rotavirus) or parasitic infection (e.g. Giardia, Cryptosporidium, Ascaris, Eimeria or Trichinella). Typically, the at least one symptom is poor stool consistency, diarrhoea or faecal blood, including faecal occult blood. Thus, methods of the present disclosure may prove effective, for example, for travellers, as a preventative or treatment for, or to reduce the severity of food poisoning.

Embodiments of the present disclosure provide methods for inhibiting or reducing inflammation or symptoms of inflammatory and autoimmune conditions, skin and nail conditions or infections and gastrointestinal infections. The term “inhibiting” and variations thereof such as “inhibition”, “inhibits”, “reduces”, “reducing” and the like, are used interchangeably herein to denote an improvement (i.e., reduction) in the severity of inflammation, or in the severity of the condition, or in the severity of infection, or in at least one symptom of the inflammation, condition or infection.

Also provided herein are methods for promoting wound healing, comprising administering to a subject in need thereof a combination of two or more strains of Lactobacillus selected from Lactobacillus buchneri, Lactobacillus zeae, Lacobacillus paracasei, Lactobacillus parafarraginis, and Lactobacillus rapi, and/or one or more culture supernatants or cell free filtrates derived from culture media in which one or more of said strains has been cultured.

Also provided herein are methods for promoting wound healing, comprising administering to a subject in need thereof a combination of microbial strains comprising strains of Lactobacillus buchneri and Lactobacillus zeae, and/or one or more culture supernatants or cell free filtrates derived from culture media in which one or more of said strains has been cultured.

Also provided herein are methods for promoting wound healing, comprising administering to a subject in need thereof a combination of microbial strains comprising strains of Lactobacillus buchneri, Lactobacillus zeae and Lactobacillus paracasei, and/or one or more culture supernatants or cell free filtrates derived from culture media in which one or more of said strains has been cultured.

Also provided herein are methods for promoting wound healing, comprising administering to a subject in need thereof a combination of microbial strains comprising strains of Lactobacillus parafarraginis, Lactobacillus buchneri, Lactobacillus rapi, Lactobacillus zeae, Acetobacter fabarum and Candida ethanolica, and/or one or more culture supernatants or cell free filtrates derived from culture media in which one or more of said strains has been cultured.

As used herein in the context of wound healing, the terms “promoting”, “promotion” and variations thereof refer to the ability of a combination or composition disclosed herein to induce, enhance or otherwise advance the natural processes associated with wound healing and/or tissue regeneration associated therewith. In embodiments the promotion may be relative to the healing observed in the absence of administration of the combination or composition. The promotion may be direct or indirect. It will be understood that in indirectly promoting wound healing, the combination or composition may effect the expression or activity of molecules which themselves regulate or otherwise influence, either directly or indirectly, the wound healing or tissue regeneration processes. The promotion may be qualitative, quantitative and/or temporal. That is, for example, the administration of the combination or composition may result in more rapid wound healing and/or tissue regeneration than would occur in the absence of such administration.

The wound may be, for example, a surgical wound, incision or superficial wound such as a cut, graze, contusion or bruise.

In particular aspects and embodiments of the present disclosure, methods comprise the administration of two or more strains selected from Lactobacillus parafarraginis, Lactobacillus buchneri, Lactobacillus rapi, Lactobacillus zeae, Lactobacillus paracasei, Acetobacter fabarum and Candida ethanolica, and/or one or more culture supernatants or cell free filtrates derived from culture media in which one or more of said strains has been cultured.

The Lactobacillus parafarraginis strain may be Lactobacillus parafarraginis Lp18. In a particular embodiment the Lactobacillus parafarraginis strain is Lactobacillus parafarraginis Lp18 deposited with National Measurement Institute, Australia on 27 Oct. 2011 under Accession Number V11/022945. This strain has been previously described in WO2013/063658.

The Lactobacillus buchneri strain may be Lactobacillus buchneri Lb23. In a particular embodiment the Lactobacillus buchneri strain is Lactobacillus buchneri Lb23 deposited with National Measurement Institute, Australia on 27 Oct. 2011 under Accession Number V11/022946. This strain has been previously described in WO2013/063658.

The Lactobacillus rapi strain may be Lactobacillus rapi Lr24. In a particular embodiment the Lactobacillus rapi strain is Lactobacillus rapi Lr24 deposited with National Measurement Institute, Australia on 27 Oct. 2011 under Accession Number V11/022947. This strain has been previously described in WO2013/063658.

The Lactobacillus zeae strain may be Lactobacillus zeae Lz26. In a particular embodiment the Lactobacillus zeae strain is Lactobacillus zeae Lz26 deposited with National Measurement Institute, Australia on 27 Oct. 2011 under Accession Number V11/022948. This strain has been previously described in WO2013/063658.

The Lactobacillus paracasei may be Lactobacillus paracasei Lp9. In a particular embodiment the Lactobacillus paracasei strain is Lactobacillus paracasei Lp9 deposited with the National Measurement Institute, Australia on 14 Dec. 2012 under Accession Number V12/022849. This strain has been previously described in WO2014/172758 (designated as strain ‘T9’ therein).

The Acetobacter fabarum strain may be Acetobacter fabarum Af15. In a particular embodiment the Acetobacter fabarum strain is Acetobacter fabarum Af15 deposited with the National Measurement Institute, Australia on 27 Oct. 2011 under Accession Number V11/022943. This strain has been previously described in WO2013/063658.

The Candida ethanolica strain may be Candida ethanolica Ce31. In a particular embodiment the Candida ethanolica strain is Candida ethanolica Ce31 deposited with the National Measurement Institute, Australia on 27 Oct. 2011 under Accession Number V11/022944. This strain has been previously described in WO2013/063658.

The method may further comprise the administration of one or more additional strains selected from Lactobacillus brevis, Lactobacillus diolivorans, Lactobacillus casei, and a strain designated herein ‘TB’ deposited with the National Measurement Institute, Australia on 14 Dec. 2012 under Accession Number V12/022850 (previously described in WO2014/172758), or one or more culture supernatants or cell free filtrates derived from culture media in which one or more of said strains have been cultured.

The concentrations of individual microbial strains to be administered in accordance with the present disclosure will depend on a variety of factors including the identity and number of individual strains employed, the exact nature and severity of the inflammation, condition or infection to be treated, the form in which a composition is applied and the means by which it is applied. For any given case, appropriate concentrations may be determined by one of ordinary skill in the art using only routine experimentation. By way of example only, the concentration of each strain present in a combination or composition may be from about 1×10² cfu/ml to about 1×10¹⁰ cfu/ml, and may be about 1×10³ cfu/ml, about 2.5×10³ cfu/ml, about 5×10³ cfu/ml, 1×10⁴ cfu/ml, about 2.5×10⁴ cfu/ml, about 5×10⁴ cfu/ml, 1×10⁵ cfu/ml, about 2.5×10⁵ cfu/ml, about 5×10⁵ cfu/ml, 1×10⁶ cfu/ml, about 2.5×10⁶ cfu/ml, about 5×10⁶ cfu/ml, 1×10⁷ cfu/ml, about 2.5×10⁷ cfu/ml, about 5×10⁷ cfu/ml, 1×10⁸ cfu/ml, about 2.5×10⁸ cfu/ml, about 5×10⁸ cfu/ml, 1×10⁹ cfu/ml, about 2.5×10⁹ cfu/ml, or about 5×10⁹ cfu/ml. In particular exemplary embodiments the final concentration of the Lactobacillus strains may be about 2.5×10⁵ cfu/ml, the final concentration of Acetobacter fabarum may be about 1×10⁶ cfu/ml and the final concentration of Candida ethanolica may be about 1×10⁵ cfu/ml.

In an exemplary embodiment, the final concentration of microbial strains to be administered in a combination or composition of strains may be between about 10⁶ and 10⁹ cfu/ml.

Also contemplated by the present disclosure is the use of variants of the microbial strains described herein. As used herein, the term “variant” refers to both naturally occurring and specifically developed variants or mutants of the microbial strains disclosed and exemplified herein. Variants may or may not have the same identifying biological characteristics of the specific strains exemplified herein, provided they share similar advantageous properties in terms of treating or preventing inflammatory skin conditions. Illustrative examples of suitable methods for preparing variants of the microbial strains exemplified herein include, but are not limited to, gene integration techniques such as those mediated by insertional elements or transposons or by homologous recombination, other recombinant DNA techniques for modifying, inserting, deleting, activating or silencing genes, intraspecific protoplast fusion, mutagenesis by irradiation with ultraviolet light or X-rays, or by treatment with a chemical mutagen such as nitrosoguanidine, methylmethane sulfonate, nitrogen mustard and the like, and bacteriophage-mediated transduction. Suitable and applicable methods are well known in the art and are described, for example, in J. H. Miller, Experiments in Molecular Genetics, Cold Spring Harbor Laboratory Press, Cold Spring Harbor, N.Y. (1972); J. H. Miller, A Short Course in Bacterial Genetics, Cold Spring Harbor Laboratory Press, Cold Spring Harbor, N.Y. (1992); and J. Sambrook, D. Russell, Molecular Cloning: A Laboratory Manual, 3rd ed., Cold Spring Harbor Laboratory Press, Cold Spring Harbor, N.Y. (2001), inter alia.

Also encompassed by the term “variant” as used herein are microbial strains phylogenetically closely related to strains disclosed herein and strains possessing substantial sequence identity with the strains disclosed herein at one or more phylogenetically informative markers such as rRNA genes, elongation and initiation factor genes, RNA polymerase subunit genes, DNA gyrase genes, heat shock protein genes and recA genes. For example, the 16S rRNA genes of a “variant” strain as contemplated herein may share about 85%, 86%, 87%, 88%, 89%, 90%, 91%, 92%, 93%, 94%, 95%, 96%, 97%, 98% or 99% sequence identity with a strain disclosed herein.

In accordance with the present disclosure, the combination to be administered may comprise one or more culture supernatants or cell free filtrates derived from culture media in which one or more of the strains has been cultured. Thus, the combination may comprise a combination of one or more strains and one or more culture supernatants or cell free filtrates derived from culture media, or may comprise one or more culture supernatants or cell free filtrates derived from culture media in which the strains have been cultured (either independently or together) in place of said strains.

The microbial strains described herein, and combinations thereof, are typically administered in accordance with the present disclosure in the form of a composition. The present disclosure makes particular reference to such compositions, and the administration of such compositions, comprising a combination of the microbial strains described herein. However those skilled in the art will appreciate that each of the microbial strains to be administered need not be contained in the same composition. Where administration is separate, administration may be sequential or simultaneous.

Compositions for use in accordance with the present disclosure may be prepared by admixing the relevant components and formulating the resulting mixture into a dosage form that is suitable for administration to a subject. Accordingly, the compositions may comprise pharmaceutically acceptable carriers, diluents, excipients and/or adjuvants. The carriers, diluents, excipients and adjuvants must be “acceptable” in terms of being compatible with other components of the composition, and not deleterious to the subject who is to receive the composition. Methods for preparing suitable compositions for administration, and carriers, diluents, excipients and adjuvants suitable for use in compositions formulated for topical administration are well known to those skilled in the art. In exemplary embodiments, the composition is formulated with a carrier comprising sterile isotonic saline or 3% sucrose.

Strains and compositions may be administered via any convenient or suitable route, variety of routes including, but not limited to, oral, rectal, topical, intranasal, intraocular, transmucosal, intestinal, enteral, intramuscular, subcutaneous, intramedullary, intrathecal, intraventricular, intracerebral, intravesical, intravenous or intraperitoneal, wherein the route of administration is not intra-vaginal. The strains and compositions may be administered in any suitable form, typically solid or liquid form. For example, the strains and compositions may be formulated, using methods and techniques well known to those skilled in the art, into tablets, troches, capsules, elixirs, suspensions, syrups, wafers, granules, powders, gels, pastes, solutions, suspensions, soluble sachets, caplets, lozenges, effervescent tablets, chewable tablets, multi-layer tablets, and the like. For oral administration, strains and compositions may be conveniently incorporated in a variety of beverages, food products, nutraceutical products, nutritional supplements, food additives, pharmaceuticals, over-the-counter formulations and animal feed supplements. For topical application, suitable vehicles include, but are not limited to, lotions, liniments, gels, creams, ointments, foams, sprays, oils, powders and the like. Compositions may also be impregnated into transdermal patches, plasters, and wound dressings such as bandages or hydrocolloid dressings, typically in liquid or semi-liquid form.

As will be appreciated by those skilled in the art, the choice of pharmaceutically acceptable carrier or diluent will be dependent on the route of administration and on the nature of the condition and the subject to be treated. The particular carrier or delivery system and route of administration may be readily determined by a person skilled in the art. A person skilled in the art will readily be able to determine appropriate formulations useful in the methods of the disclosure using conventional approaches.

For example, compositions useful in the methods of the present disclosure may be formulated for administration in the form of liquids, containing acceptable diluents (such as saline and sterile water), or may be in the form of lotions, creams or gels containing acceptable diluents or carriers to impart the desired texture, consistency, viscosity and appearance. Acceptable diluents and carriers are familiar to those skilled in the art and include, but are not restricted to, ethoxylated and nonethoxylated surfactants, fatty alcohols, fatty acids, hydrocarbon oils (such as palm oil, coconut oil, and mineral oil), cocoa butter waxes, silicon oils, pH balancers, cellulose derivatives, emulsifying agents such as non-ionic organic and inorganic bases, preserving agents, wax esters, steroid alcohols, triglyceride esters, phospholipids such as lecithin and cephalin, polyhydric alcohol esters, fatty alcohol esters, hydrophilic lanolin derivatives and hydrophilic beeswax derivatives.

Alternatively, the microbial strains can be formulated readily using pharmaceutically acceptable carriers well known in the art into dosages suitable for oral administration. These carriers may be selected from sugars, starches, cellulose and its derivatives, malt, gelatin, talc, calcium sulfate, vegetable oils, synthetic oils, polyols, alginic acid, phosphate buffered solutions, emulsifiers, isotonic saline and pyrogen-free water.

For compositions formulated for topical administration, examples of pharmaceutically acceptable diluents are demineralised or distilled water; saline solution; vegetable based oils such as peanut oil, safflower oil, olive oil, cottonseed oil, maize oil, sesame oils such as peanut oil, safflower oil, olive oil, cottonseed oil, maize oil, sesame oil, arachis oil or coconut oil; silicone oils, including polysiloxanes, such as methyl polysiloxane, phenyl polysiloxane and methylphenylpolysolpoxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxypropylmethylcellulose; lower alkanols, for example ethanol or iso-propanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrridone; agar; carrageenan; gum tragacanth or gum acacia, and petroleum jelly.

In further embodiments, the composition may further comprise suspending agents and/or humectants, such as povidone or propylene glycol, and neutralising agents for adjusting the viscosity of the composition, such as sodium hydroxide, triethanolamine (TEA) or ethylenediamine tetraacetic acid (EDTA).

Strains and compositions may be administered on, for example, a once-a-day basis or alternatively on multiple occasions per day depending on the desired outcome. The amount of extract or composition to be administered, and the frequency of administration will vary depending on a range of factors including the identity of the strains, the nature and severity of the condition suffered by the subject, the age and general wellbeing of the subject, and the desired therapeutic outcome. Suitable dosage regimes can readily be determined by the skilled addressee.

In exemplary embodiments, an about 0.25 ml to about 10 ml liquid formulation of a combination of microbial strains at a final concentration of between about 10⁶ and 10⁸ cfu/ml may be orally administered to a human subject on a once-a-day, twice-a-day or more frequent basis. The volume of the liquid formulation may be about 0.25 ml, 0.5 ml, 0.75 ml, 1 ml, 1.25 ml, 1.5 ml, 1.75 ml, 2 ml, 2.25 ml, 2.5 ml, 2.75 ml, 3 ml, 3.5 ml, 4 ml, 4.5 ml, 5 ml, 5.5 ml, 6 ml, 6.5 ml, 7 ml, 7.5 ml, 8 ml, 8.5 ml, 9 ml, 9.5 ml, or 10 ml. In a particularly exemplary embodiment the liquid formulation for human use is an approximately 1 ml to 2 ml formulation comprising about 10⁶ to 10⁹ cfu/ml of the microbial strains.

In other exemplary embodiments, an about 1 ml to about 25 ml liquid formulation of a combination of microbial strains at a final concentration of between about 10⁶ and 10⁸ cfu/ml may be orally administered to a livestock animal subject on a once-a-day, twice-a-day or more frequent basis. The volume of the liquid formulation may be about 1 ml, 2 ml, 3 ml, 4 ml, 5 ml, 6 ml, 7 ml, 8 ml, 9 ml, 10 ml, 11 ml, 12 ml, 13 ml, 14 ml, 15 ml, 16 ml, 17 nil, 18 ml, 19 ml, 20 nil, 21 ml, 22 nil, 23 nil, 24 ml, or 25 ml. In a particularly exemplary embodiment the liquid formulation for livestock use is an approximately 10-15 ml formulation comprising about 10⁶ to 10⁸ cfu/ml of the microbial strains.

The microbial strains may be combined with other therapeutic or cosmetic agents for example, but not limited to, antibiotics, antimicrobial agents, antiseptics, anaesthetics, anti-infective agents, anti-inflammatory agents, and other therapeutic agents indicated for the treatment of inflammatory conditions such as steroids, and NSAIDs. Administration of such additional agents may be at the same time or at different times, i.e. simultaneous or sequential, and may be administered by the same or different routes, with respect to the one or more microbial strains the subject of the present disclosure. Additional therapeutic agents may be co-formulated with microbial strains used in the methods.

Non-limiting examples of additional anti-inflammatory agents that may be employed include steroidal and non-steroidal compounds such as clobetasol propionate, betamethasone dipropionate, halobetasol proprionate, diflorasone diacetate, fluocinonide, halcinonide, amcinonide, desoximetasone, triamcinolone acetonide, mometasone furoate, fluticasone propionate, betamethasone dipropionate, fluocinolone acetonide, hydrocortisone valerate, hydrocortisone butyrate, flurandrenolide, triamcinolone acetonide, mometasone furoate, triamcinolone acetonide, fluticasone propionate, desonide, fluocinolone acetonide, hydrocortisone valerate, prednicarbate, triamcinolone acetonide, desonide, hydrocorti-sone, hydrocortisone aceponate, hydrocortisone buteprate, methylprednisolone aceponate, mometasone furoate and prednicarbate. Non-limiting examples of suitable non-steroidal anti-inflammatory compounds include indomethacin, ketoprofen, felbinac, diclofenac, ibuprofen, piroxicam, benzydamin, acetylsalicylic acid, diflunisal, salsalate, naproxen, fenoprofen, ketoprofen, flurbiprofen, oxaprozin, loxoprofen, indomethacin, sulindac, etodolac, ketorolac, diclo-fenac, nabumetone, piroxicam, meloxicam, tenoxicam, droxicam, lornoxicam, isoxicam, mefenamic acid, meclofenamic acid, flufenamic acid, tolfenamic acid, firocoxib, and licofelone, semi-synthetic glycosaminoglycosan ethers, flavanols, flavonoids, isoflavones and derivatives.

The anti-infective agent may be any agent which treats an infection in a subject. In particular embodiments, the anti-infective agent is able to kill or inhibit the growth of an infectious organism which is capable of being transferred, in entirety or in part, between cells via an apoptotic body. Suitable anti-infective agents include, but are not limited to, an anti-viral, an anti-bacterial, an anti-protozoal, an anti-fungal or a combination thereof.

Illustrative anti-virals include, but are not limited to, abacavir sulfate, acyclovir especially acyclovir sodium, adefovir, amantadine especially amantadine hydrochloride, amprenavir, ampligen, atazanavir, cidofovir, darunavir, delavirdine especially delavirdine mesylate, didanosine, docosanol, dolutegravir, edoxudine, efavirenz, emtricitabine, elvitegravir, enfuvirtide, entecavir, famciclovir, fomivirisen especially fomivirsen sodium, foscarnet especially foscarnet sodium, ganciclovir, ibacitabine, idoxuridine, imiquimod, indinavir especially indinavir sulfate, inosine pranobex, lamivudine, lopinavir, maraviroc, metisazone, moroxydine, nelfinavir especially nelfinavir mesylate, nevirapine, nitazoxanide, oseltamivir particularly oseltamivir phosphate, penciclovir, peramivir, pleconaril, podophyllotoxin, raltegravir, ribavirin, rimantadine especially rimantadine hydrochloride, ritonavir, saquinavir especially saquinavir mesylate, sofosbuvir, stavudine, telaprivir, tenofovir, tipranovir, trifluridine, tromantadine, umifenovir, valacyclovir especially valacyclovir hydrochloride, valganciclovir, vicriviroc, vidarabine, viramidine, zalcitabine, zanamivir, zidovudine and pharmaceutically acceptable salts and combinations thereof.

Illustrative anti-bacterials include, but are not limited to, quinolones (e.g. amifloxacin, cinoxacin, ciprofloxacin, enoxacin, fleroxacin, flumequine, lomefloxacin, nalidixic acid, norfloxacin, ofloxacin, levofloxacin, lomefloxacin, oxolinic acid, pefloxacin, rosoxacin, temafloxacin, tosufloxacin, sparfloxacin, clinafloxacin, gatifloxacin, moxifloxacin, gemifloxacin, and garenoxacin), tetracyclines, glycylcyclines and oxazolidinones (e.g. chlortetracycline, demeclocycline, doxycycline, lymecycline, methacycline, minocycline, oxytetracycline, tetracycline, tigecycline; linezolide, eperezolid), glycopeptides, aminoglycosides (e.g. amikacin, arbekacin, butirosin, dibekacin, fortimicins, gentamicin, kanamycin, menomycin, netilmicin, ribostamycin, sisomicin, spectinomycin, streptomycin, tobramycin), β-lactams (e.g. imipenem, meropenem, biapenem, cefaclor, cefadroxil, cefamandole, cefatrizine, cefazedone, cefazolin, cefixime, cefmenoxime, cefodizime, cefonicid, cefoperazone, ceforanide, cefotaxime, cefotiam, cefpimizole, cefpiramide, cefpodoxime, cefsulodin, ceftazidime, cefteram, ceftezole, ceftibuten, ceftizoxime, ceftriaxone, cefuroxime, cefuzonam, cephacetrile, cephalexin, cephaloglycin, cephaloridine, cephalothin, cephapirin, cephradine, cefinetazole, cefoxitin, cefotetan, azthreonam, carumonam, flomoxef, moxalactam, amdinocillin, amoxicillin, ampicillin, azlocillin, carbenicillin, benzylpenicillin, carfecillin, cloxacillin, dicloxacillin, methicillin, mezlocillin, nafcillin, oxacillin, penicillin G, piperacillin, sulbenicillin, temocillin, ticarcillin, cefditoren, SC004, KY-020, cefdinir, ceftibuten, FK-312, S-1090, CP-0467, BK-218, FK-037, DQ-2556, FK-518, cefozopran, ME1228, KP-736, CP-6232, Ro 09-1227, OPC-20000, LY206763), rifamycins, macrolides (e.g. azithromycin, clarithromycin, erythromycin, oleandomycin, rokitamycin, rosaramicin, roxithromycin, troleandomycin), ketolides (e.g. telithromycin, cethromycin), coumermycins, lincosamides (e.g. clindamycin, lincomycin), chloramphenicol, clofazimine, cycloserine, dapsone, ethambutol hydrochloride, isoniazid, pyrazinamide, rifabutin, rifampin, rifapentine and streptomycin sulfate.

Illustrative anti-protozoals include, but are not limited to, atovaquone, metronidazole including metronidazole hydrochloride, pentamidine including pentamidine isethionate, chloroquine including chloroquine hydrochloride and chloroquine phosphate, doxycycline, hydroxychloroquine sulfate, mefloquine including mefloquine hydrochloride, primaquine including primaquine phosphate, pyrimethamine, pyrimethamine with sulfadoxine, trimethoprim, sulfamethoxazole, clindamycin, quinine, quinidine, sulfadiazine, artemether, lumefantrine, artesunate, nitazoxanide, suramin, melarsoprol, eflornithine, nifurtimox, stibogluconate including sodium stibogluconate, amphotericin B including liposomal amphotericin B, miltefosine, paromomycin, ketoconazole, itraconazole, fluconazole, and pharmaceutically acceptable salts and combinations thereof.

Illustrative anti-fungals include, but are not limited to, abafungin, albaconazole, amorolfine, amphotericin B, amphotericin B cholesteryl sulfate complex, amphotericin B lipid complex, amphotericin B liposomal, anidulafungin, bifonazole, butenafine, butoconazole, candicidin, caspofungin, clotrimazole, econazole, efinaconazole, fenticonazole, fluconazole, flucytosine, griseofulvin microsize, griseofulvin ultramicrosize, hamycin, isavuconazole, isoconazole, itraconazole, ketoconazole, luliconazole, micafungin, miconazole, naftifine, natamycin, nystatin, omoconazole, oxiconazole, posaconazole, propiconazole, ravuconazole, sertaconazole, sulconazole, terbinafine including terbinafine hydrochloride, terconazole, tioconazole, voriconazole, and pharmaceutically acceptable salts and combinations thereof.

In exemplary embodiments the combinations of microbial strains described herein are provided in the form of probiotic compositions. Such compositions may further comprise one or more additional probiotic microorganisms such as, for example, Lactobacillus rhamnosus, Lactobacillus plantarum, Lactobacillus bulgaricus, Lactobacillus paracasei, Lactobacillus casei, Lactobacillus acidophilus, Lactobacillus fermentum, Lactococcus lactis, Streptococcus thermophilus, Bifidobacterium breve, Bifidobacterium bifidum, Bifidobacterium animalis subsp. lactis, and Bifidobacterium animalis subsp. animalis.

Probiotic compositions may comprise one or more prebiotic components. Suitable prebiotics include, for example, polydextrose, inulin, fructooligosaccharides (FOS), xylooligosaccharides (XOS), galactooligosaccharides (GOS), mannan oligosaccharides, protein-based green lipped mussel extract, and various prebiotic-containing foods such as raw onion, raw leek, raw chickory root and raw artichoke. In certain embodiments the prebiotic is a fructooligosaccharide.

Compositions comprising microbial combinations described herein may be administered in any suitable form, including any of the dosage forms described above. The probiotic compositions may be provided to the user in a powder form, suitable for mixing by the user into any type of drink or food product (for example water, fruit juice or yoghurt) or for consumption as a powder in the absence of a drink or additional food product. The probiotic compositions may therefore be conveniently incorporated in a variety of food and/or beverage products, nutriceutical products, probiotic supplements, food additives, and over-the-counter formulations. The food or food additive may be a solid form such as a powder, or a liquid form. Specific examples of the types of beverages or foods include, but are not limited to water-based, milk-based, yoghurt-based, other dairy-based, milk-substitute based such as soy milk or oat milk, or juice-based beverages, water, soft drinks, carbonated drinks, and nutritional beverages, (including a concentrated stock solution of a beverage and a dry powder for preparation of such a beverage); baked products such as crackers, breads, muffins, rolls, bagels, biscuits, cereals, bars such as muesli bars, health food bars and the like, dressings, sauces, custards, yoghurts, puddings, pre-packaged frozen meals, soups and confectioneries.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

The present disclosure will now be described with reference to the following specific examples, which should not be construed as in any way limiting the scope of the invention.

### EXAMPLES

The following examples are illustrative of the invention and should not be construed as limiting in any way the general nature of the disclosure of the description throughout this specification.

### Example 1—Human Case Studies

### Case Study 1

Following radiation therapy the subject began to experience gum infections and was prescribed an antibiotic. However upon taking the antibiotic the subject's skin erupted into lesions and a rash affecting most of the body, although most prominent on the legs. After a few months dermatitis with scaling appeared, together with general swelling. In the space of six months treatments applied included antihistamines, four courses of antibiotics, cortisone ointment (twice a day), moisturisers and oil baths. While the antibiotics appeared to provide some temporary relief, after each course of antibiotic was finished, the skin flared up again with lesions, welts and a rash.

A liquid composition comprising a combination of Lactobacillus parafarraginis Lp18, Lactobacillus buchneri Lb23, Lactobacillus rapi Lr24, Lactobacillus zeae Lz26, Acetobacter fabarum Af15 and Candida ethanolica Ce31 was applied to the subject's ankles where the skin lesions, welts and rash were most problematic. Cottonballs were soaked in the liquid composition then held in contact with the ankle overnight using plastic film. There was an immediate result. Prior to the initial treatment with the liquid composition the whole ankle area was bright red with layers of scaling skin. The first morning after the initial treatment with the liquid composition the redness had reduced and the scaling skin was gone. Several raised areas around 2 cm in diameter indicated the source of the problem. Subsequent treatment with the liquid composition was then restricted to these areas, which subsided day by day until they had disappeared in less than two weeks. Over the same time period the rash and swelling disappeared.

### Case Study 2

A 55 year old male presented with joint pain from hip and knee operations resulting in difficulty walking distances, especially up hill or stairs. The subject placed 2 ml of a liquid formulation comprising a combination of Lactobacillus buchneri Lb23, Lactobacillus zeae Lz26 and Lactobacillus paracasei Lp9 under the tongue for several minutes before swallowing, on a daily basis. The formulation comprised about 10⁶ to 10⁸ cfu/ml of the microbial strains in sterile saline and 2-3% sucrose (stored at 4° C. until used).

After 2 to 3 days, joint pain had subsided and the subject now has freedom from pain while exercising and walking up stairs. Following treatment, his movement when walking up stairs no longer requires him to take one step then lift the injured leg up to the same step before taking the next step.

### Case Study 3

A 53 year old male was diagnosed with osteoarthritis in the right knee, back pain from wear and tear, pain soreness and inflammation from general home duties including gardening and exercising. He was unable to perform these normal activities for any period of time without being bed ridden or very sore the next day. The subject took 2 ml of the liquid formulation described above for Case Study 2 on a daily basis. Following treatment he is free of pain and can resume general activities including gardening and exercising with out pain or discomfort. He also reported that his joints and back have a wider range of movement than prior to treatment.

### Case Study 4

A 58 year old male had surgical treatment on a leg. After administration of 2 ml of the formulation described above for Case Study 2 for several days, the subject reported that the wound healed much faster than was expected by his physician. Additionally, his joint pain from a knee operation and osteoarthritis of the hip were improved such that he reported freedom of movement free from joint pain when operating on the family farm.

### Case Study 5

A 40 year old female had digestive issues and longer term irritable bowel syndrome. After taking 2 ml of the formulation described above for Case Study 2 under the tongue daily for 3 days, she reported less digestive tract problems and an immediate improvement in general wellbeing. Her ability to control her bowel problems significantly improved and bowel movements were more regular.

### Case Study 6

A 53 year old male reported continual swelling and soreness of both knees due to playing elite level sport that resulted in lateral meniscectomy and medial meniscectomy and ACL ligament damage. Suspected osteoarthritis. After taking 2 ml of the formulation described above for Case Study 2 under the tongue daily for two weeks, he reported reduced swelling and less joint pain. Walking longer distances on hard surfaces improved and general knee areas were less painful with less aches at the days end.

### Case Study 7

A 34 year old female suspected of having irritable bowel syndrome reported a 50-70% reduction in symptoms (less stomach pain, flatulence and greater bowel regulatory) over the course of a 4 week treatment regime comprising 2 ml of the formulation described above for Case Study 2 taken under the tongue daily.

### Case Study 8

A 35 year old male presented with inflammation and pain (rating 6/10) in his right knee due to a previous injury (meniscus and ligament damage). The subject took 2 ml of the formulation described above for Case Study 2 per day by mouth in the morning for 5 consecutive days (total accumulative dose=10 ml). Symptoms improved after 3 days, pain diminished to a negligible level (rating 1/10) and he has little discomfort. Additionally, at the time he started treatment with the formulation he had some ear, nose and throat inflammation suspected to be due to a low level chronic infection. The inflammation and swelling was periodically quite painful (rating 5/10) and fluid clearance troublesome. During and after taking the formulation the subject observed a decrease in pain (to a rating of 1/10) and inflammation, and reported better mucosal clearance. The subject felt that his overall level of health had improved and reported an improvement in his overall wellbeing.

### Case Study 9

A 30 year old female subject presented with eczema on her scalp and back. Following several days of topical administration of a formulation as described above for Case Study 2, the subject's lesions cleared up.

### Case Study 10

A 43 year old male presented with tinea pedis between toes on both feet. The subject topically applied 0.75 ml to 1 ml of the formulation described above for Case Study 2 per foot, rubbed lightly across affected area, daily. Symptoms completely disappeared within 2 to 3 days, and application of the formulation was ceased after 7 days.

### Case Study 11

A 43 year old female presented with toe nail fungus affecting the large toe on one foot. About 1 ml of the formulation described above for Case Study 2 was applied topically daily, by rubbing over the affected nail, the cuticle and the skin slightly above the nail. Symptoms of the fungal infection completely disappeared within 7 days.

### Example 2—Animal Case Studies

### Case Study 1

A dairy cow suffered an injured udder from being stood on in the dairy. The cow was treated with 15 ml of a formulation comprising a combination of Lactobacillus buchneri Lb23, Lactobacillus zeae Lz26 and Lactobacillus paracasei Lp9 orally on a daily basis. The formulation comprised about 10⁶ to 10⁸ cfu/ml of the microbial strains in sterile saline and 2-3% sucrose (stored at 4° C. until used).

The veterinarian observed that the bruising and swelling subsided faster than would have been expected in the absence of the administration of the formulation, and the cow resumed normal feeding with 24 to 48 hrs of treatment.

### Case Study 2

Calves often experience digestive issues such as scours (non-specific intestinal problems) and can deteriorate to the point of death in 48 hrs. Treatment of calves with scours with 15 ml of the formulation as described above (animal case study 1) via drenching or via milk resulted in the calves surviving, and within 24 to 48 hrs being noticeably less stressed and back on normal feeding patterns.

### Case Study 3

A horse was in obvious distress and pain from an injured leg. The horse did not want to stand and had been off her feed. Treatment with 15 ml of the formulation as described above for animal case study 1 orally twice a day resulted the horse being noticeably more relaxed and less distressed, as indicated by standing on all legs and not laying down, within only 24 to 48 hrs. The horse resumed feeding normally within 24 hrs of the first treatment.

### Example 3—DSS-Induced Mouse Model of Acute Colitis

The inventors then examined the effect of a formulation comprising Lactobacillus buchneri Lb23, Lactobacillus zeae Lz26 and Lactobacillus paracasei Lp9 in a dextran sodium sulfate (DSS)-induced model of acute colitis in mice. The formulation comprised about 10⁶ or 10⁹ cfu/ml of the microbial strains in sterile saline and 2-3% sucrose (stored at 4° C. until used). Prior to dosing, each bacterial formulation was analysed for cell viability/count. DSS was from MP BioMedicals), stored at room temperature. On day 1 of dosing 3% DSS solution was freshly prepared by dissolving DSS in sterile water.

40 female C57BL/6NTac mice were divided into four treatment groups:

Group 1—non-treatment (negative control) group. n=10.

Group 2—3% DSS+vehicle (9% sterile saline). n=10.

Group 3—3% DSS+bacterial formulation at 10⁶ cfu/ml. n=10.

Group 4—3% DSS+bacterial formulation at 10⁹ cfu/ml. n=10.

Animals of Groups 2 to 4 received 3% DSS ad libitum via sterile drinking water on days 1 to 5, 13 to 17 and 25 to 29, while Group 1 animals continued to receive only sterile water as drinking water. Animals of Groups 2 to 4 received either 1 ml vehicle, 1 ml bacterial formulation at 10⁶ cfu/ml or 1 ml bacterial formulation at 10⁹ cfu/ml, respectively, once daily by oral gavage.

Symptoms of DSS-induced colitis (stool consistency and faecal blood occurrence) were assessed by measuring in-life endpoints from days 1 to 29, every other day. On days of dosing, evaluations were performed 1 to 2 hours after dosing.

Stool was collected from day 1 for each mouse and examined for consistency. Stool consistency was graded as follows: normal=0; soft, but still formed=1; very soft=2; diarrhoea=4. Faecal blood in the stool was detected using the Hemoccult Tape Test Kit (Beckman Coulter, according to manufacturer's instructions). Scoring for faecal blood was as follows: negative hemoccult=0; positive hemoccult (slight colour on strip)=1; positive hemoccult (darker colour on strip)=2; visible traces of blood=3; gross rectal bleeding=4.

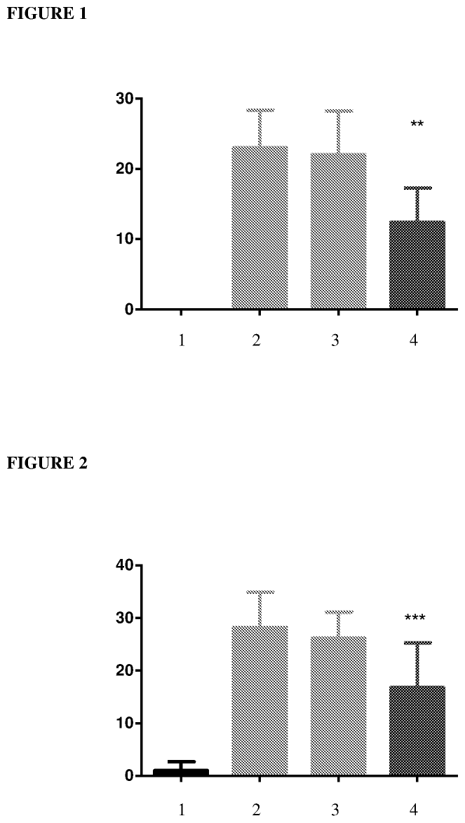

As shown in FIG. 1, over days 1 to 11 of treatment, faecal blood occurrence was significantly reduced (p<0.01) in mice of treatment Group 4 compared to Group 2. Similarly, the combination of stool consistency and faecal blood occurrence was significantly improved (p<0.001) in mice of treatment Group 4 compared to Group 2. There was no significant difference in body weight between the groups over the course of the study.

1. A method for treating or preventing inflammation, an inflammatory or autoimmune condition, or a skin or nail condition or infection in a subject, the method comprising administering to the subject a combination of two or more strains of Lactobacillus selected from Lactobacillus buchneri, Lactobacillus zeae, Lacobacillus paracasei, Lactobacillus parafarraginis, and Lactobacillus rapi, and/or one or more culture supernatants or cell free filtrates derived from culture media in which one or more of said strains has been cultured.

2. (canceled)

3. A method according to claim 1, comprising administering a combination of strains of two, three, four or all of said Lactobacillus species, and/or one or more culture supernatants or cell free filtrates derived from culture media in which one or more of said strains has been cultured.

4. A method according to claim 1, comprising administering a combination of Lactobacillus buchneri and Lactobacillus zeae, and/or one or more culture supernatants or cell free filtrates derived from culture media in which one or more of said strains has been cultured.

5. A method according to claim 1, comprising administering a combination of Lactobacillus buchneri, Lactobacillus zeae and Lacobacillus paracasei, and/or one or more culture supernatants or cell free filtrates derived from culture media in which one or more of said strains has been cultured.

6. A method according to claim 1, further comprising administering a strain of Acetobacter fabarum or a culture supernatant or cell free filtrate derived from culture media in which said strain has been cultured, and/or a strain of Candida ethanolica or a culture supernatant or cell free filtrate derived from culture media in which said strain has been cultured.

7. A method according to claim 1, comprising administering combination of Lactobacillus parafarraginis, Lactobacillus buchneri, Lactobacillus rapi, Lactobacillus zeae, Acetobacter fabarum and Candida ethanolica, and/or one or more culture supernatants or cell free filtrates derived from culture media in which one or more of said strains has been cultured.

8. A method according to claim 1, wherein the inflammation is gastrointestinal inflammation, or inflammation of the joints.

9. A method according to claim 8, wherein the gastrointestinal inflammation comprises gastritis or gastroenteritis.

10. (canceled)

11. A method according to claim 8, wherein the inflammation of the joints comprises, or is associated with, arthritis.

12. A method according to claim 11, wherein the arthritis is rheumatoid arthritis or osteoarthritis.

13. A method according to claim 1, wherein the inflammatory or autoimmune condition is a gastrointestinal condition, or a skin or nail condition or infection.

14. A method according to claim 13, wherein the gastrointestinal condition is an inflammatory bowel disease, a gastrointestinal infection, or is associated with a gastrointestinal infection.

15. A method according to claim 14, wherein the inflammatory bowel disease is ulcerative colitis, Crohn's disease or irritable bowel syndrome.

16. (canceled)

17. (canceled)

18. A method according to claim 1, wherein the skin condition is psoriasis, dermatitis, eczema, rosacea, acne, or ichtyosis.

19. A method according to claim 1, wherein the skin or nail infection is a fungal infection.

20. A method according to claim 19, wherein the fungal infection is tinea pedis (athlete's foot), tinea cruris (jock itch), tinea capitis, tinea corporis or tinea unguium (onychomycosis).

21. A method for preventing or treating at least one symptom of a gastrointestinal infection, the method comprising administering to a subject in need thereof a combination of two or more strains of Lactobacillus selected from Lactobacillus buchneri, Lactobacillus zeae, Lacobacillus paracasei, Lactobacillus parafarraginis, and Lactobacillus rapi, and/or one or more culture supernatants or cell free filtrates derived from culture media in which one or more of said strains has been cultured.

22. A method according to claim 21, wherein the at least one symptom is poor stool consistency, diarrhoea or faecal blood.

23. A method for promoting wound healing in a subject, the method comprising administering to the subject:
a combination of two or more strains of Lactobacillus selected from Lactobacillus buchneri, Lactobacillus zeae, Lacobacillus paracasei, Lactobacillus parafarraginis, and Lactobacillus rapi, and/or one or more culture supernatants or cell free filtrates derived from culture media in which one or more of said strains has been cultured;
a combination of microbial strains comprising strains of Lactobacillus buchneri and Lactobacillus zeae, and/or one or more culture supernatants or cell free filtrates derived from culture media in which one or more of said strains has been cultured;
a combination of microbial strains comprising strains of Lactobacillus buchneri, Lactobacillus zeae and Lactobacillus paracasei, and/or one or more culture supernatants or cell free filtrates derived from culture media in which one or more of said strains has been cultured; or
a combination of microbial strains comprising strains of Lactobacillus parafarraginis, Lactobacillus buchneri, Lactobacillus rapi, Lactobacillus zeae, Acetobacter fabarum and Candida ethanolica, and/or one or more culture supernatants or cell free filtrates derived from culture media in which one or more of said strains has been cultured.

24. (canceled)

25. (canceled)

26. (canceled)

27. A method according to claim 23, wherein the wound is a surgical wound.
